(19) United States
(12) Patent Application Publication
(10) Pub. No.: US 2017/0196923 A1
(43) Pub. Date: Jul. 13, 2017

(54) Cannabis-Hemp Paste

(71) Applicant: David Harold Moore, Henderson, NV (US)

(72) Inventor: David Harold Moore, Henderson, NV (US)

(21) Appl. No.: 14/969,497

(22) Filed: Jan. 12, 2016

(51) Int. Cl. A61K 36/185

(52) U.S. Cl. CPC A61K 36/185; A23L 1/3002; A23V 2002/00

(57) ABSTRACT

The present invention provides a commercial process for preparing the ‘Cannabis-Hemp Paste’ into a food grade nutritional supplement. The process produces both a fully cooked, and raw food product combined, that can be inserted into an edible consumption vessel of any type, or placed into the mouth, and/or stomach, for ingestion purposes.

The commercial process entails the combining of various terpenes, flavonoids, decarboxylated cannabinoids (and various raw cannabinoids) that are found in the Cannabis-Hemp Plant; with various fats and thickeners for the purposes of creating a ‘paste’ that can be ingested and metabolized as a food grade dietary supplement, and/or as a direct food product.

To accomplish this process, a quantity of the Cannabis-Hemp Plant must be raw and pressed, and a quantity of the Cannabis-Hemp Plant must be heat-cooked and blended with fresh fats; the heat-cooked Cannabis-Hemp plant must then be repeatedly heat-cooked in a steam kettle and pressed to extract different amalgams.

### BACKGROUND

My name is David Harold Moore and I am the inventor of the ‘Cannabis Hemp Paste’ process. I have suffered from a High Functioning form of Autism, known as Asperger's Syndrome, since I was born. The autism caused the dysregulation of my endocannabinoid immune system, which caused the auto-immune disorder known as Celiac Disease, which then turned into stomach cancer. And only after going into renal failure, did I begin to look for a solution:
Cannabinoids as Antioxidants and Neuroprotectants
U.S. Pat. No. 6,630,507 B1
Abstract
Cannabinoids have been found to have antioxidant properties, unrelated to NMDA receptor antagonism. This newfound property makes cannabinoids useful in the treatment and prophylaxis of wide variety of oxidation associated diseases, such as ischemic, age-related, inflammatory and autoimmune diseases. The cannabinoids are found to have particular application as neuroprotectants, for example in limiting neurological damage following ischemic insults, such as stroke and trauma, or in the treatment of neurodegenerative diseases, such as Alzheimer's disease, Parkinson's disease and HIV dementia. Nonpsychoactive cannabinoids, such as cannabidoil (CBD), are particularly advantageous to use . . .

So after doing a little bit of research, I read the patent above, and I also found out that half the fat in coconut-oil is comprised of a fat that is not frequently found in nature, lauric-acid. Lauric-acid has been found to promote health, and is also present in a mother's breast milk. In fact, it can be found in two main dietary sources: small amounts in butterfat, and larger amounts in coconut oil.

In the body lauric-acid is converted to monolaurin, which is a potent antiviral, antibacterial and antiprotozoal substance. Because monolaurin is a monoglyceride, it can destroy lipid-coated viruses including: measles, influenza, HIV, herpes, and a number of pathogenic bacteria . . . with the Cannabis-Hemp Plant providing cannabinoids to help up-regulate the immune dysregulation that occurs as a result of the autistic brain, as well as improving glycemic control, and killing cancer (to name a few).

Then I read a recent study from the Biomedical Center for Autism Research and Treatment, which suggests the CB2 receptor as a potential therapeutic target for the cannabis-hemp infused coconut-oil. This study was the first to demonstrate the up-regulation of CB2 receptors in autistic endocannabinoid systems.

The CB2 receptor enhancements demonstrate that the endocannabinoid system is involved in the maintenance of autism. According to the findings, the endocannabinoid system may represent a novel treatment opportunity for cannabis therapy with autistic minds. And that's because the up-regulation of the CB2 receptors enhances the peripheral organs ability to manage the immune system. And endocannabinoid signaling, in general, affects memory formation, learning, pain, and other important regenerative processes.

You see, the endocannabinoid system is an intricate network of endocannabinoids, their receptors, and the associated enzymes; and all play critical roles in maintaining the body's homeostasis. And our biochemical homeostasis is unique to each one of us. It is therefore important to note, that in most cases, scientists make decisions based on evidence gained from an experiment with extensive controls. But the differences in cannabinoid composition, varies from batch to batch, thus making cannabis-hemp experiments too organic for scientific rigidity.

And this is where the ‘aha moment’ of idea crystallization begins to take shape. For I began to realize, after working with Cannabis-Hemp Edibles, that the food combined with the commercial edibles were not helping my illness. In fact, the sweetened edibles were causing me to become more distressed. So then I tried the commercial Cannabis-Hemp Concentrates, but they were almost all contaminated with pesticides, and most were too psychoactive for my purposes (I still needed to make a living). And none of the current commercial options were affordable for repeated daily use.

So I needed to find a healthy and inexpensive way to make a nutritional Cannabis-Hemp food grade dietary supplement. A daily dietary supplement that was all natural and healthy, a capsule with little to no psychoactivity, and a capsule that could be taken in large quantities without harmful side effects. But there were no commercial products that would meet my needs.

Then I began experimenting with different ingredients and different heating methods. This process took about two years before I came up with a novel, non-obvious and useful method to process the Cannabis-Hemp plant for a food grade dietary supplement that could be ingested several times a day.

And this novel, non-obvious and useful method is a process based upon the different pressings of the raw and cooked cannabis-hemp plant, combined with various fats, to produce different amalgams that holistically synergize to create the wholesome food product, and nutritional dietary supplement, called ‘Cannabis-Hemp Paste’.

### SUMMARY

Cannabis-Hemp Paste is a novel, non-obvious and useful method to process the Cannabis-Hemp Plant for a food grade dietary supplement.
A primary object of the present invention is to provide an inexpensive method to commercially process the Cannabis-Hemp plant into a food grade dietary supplement, so that the process overcomes the shortcomings of the other methods.
Another object is to provide a whole plant supplement to reduce the amount of wasted Cannabis-Hemp material that is being thrown away after current extraction methods.
An additional object is to provide a food product that can be stored for extended periods of time, to place that food product within a sealed capsule that doesn't leak, and to package the food product in resealable food sage bags.
Another object is to provide an affordable Cannabis-Hemp food grade dietary supplement that can be ingested several times a day.

Further objects of the invention will appear as the description proceeds.

Briefly described, in its preferred form, the present invention provides a method for preparing a commercially inexpensive way to produce a high quality Cannabis-Hemp Paste. The present process preferably comprises the following steps:
a. providing a quantity of the raw cannabis-hemp plant (food product);
b. providing a system to convey the food product through the various steps of the process;
c. heat cooking the food product;
d. blending various fats with the food product;
e. heat cooking the food product with steam;
f. amalgamating the various added fats with the cannabinoids, terpenes, terpenoids, and flavonoids;
g. pressing the cooked and raw food product to extract liquids;
h. blending the extracted liquids;
i. blending the extracted liquids with both the raw and cooked food product.

A raw food product at ambient temperature is the preferred initial food product for the present process. That is, the most desirable final Cannabis Hemp Paste product begins with a raw product in the beginning of the process. While an initially raw/fresh initial product is preferred, the present process can accommodate an initial food product that has been frozen and dried, that is above or below ambient temperature and that has already been dehumidified.

The steam and heat-cooking temperature and the time period the product stays within the cooking chamber can be adjusted for the various types of amalgams the present invention is designed to handle. Each component step of the present invention can similarly be adjusted. Some of the adjustments can be incorporated by simply manipulating heat times.

The preferable heat-cooking and steam kettle steps may comprise only a steam cooking step or a dry heat-cooking step. Yet if the product is only cooked with steam, the final product typically lacks the amount of decarboxylated cannabinoids of the both steam and heat-cooked product. If the product is only heated without added fats, and without the other steam kettle steps, the process will produce an inferior product.

In the conventional processing methods, typically the food product is concentrated to produce intense psychoactivity. One of the problems with this conventional process is that by concentrating the endocannabinoids, one also concentrates environmental pollutants and pesticides. The final product can therefore be harmful to our health, as well as impractical for daily use.

In the present process, the fats are applied after convection cooking, and a raw portion of the final food product is never heated so as to preserve the naturally occurring enzymes, terpenes, terpenoids, and flavonoids. Thus, the product retains its full compliment of healthy ingredients because different amalgams are added throughout the various heating and pressing processes to create the enhanced superior product known as ‘Cannabis Hemp Paste’.

The present Cannabis-Hemp Paste process incorporates different cooling-resting steps to cool the product down so as to encourage amalgamation during the entire process. If the food product is too hot at any point in the process, then many of the cannabinoids will be lost. If the food product is too cool during the cooking process, then the cannabinoids won't decarboxylate and the product won't be as effective.

Furthermore, present manufacturers provide an end product that is fully cooked, so when the consumer ingests the product, they don't receive the full spectrum of cannabinoids. Unlike the conventional processes, the present invention combines both raw and cooked amalgams to the final Cannabis-Hemp Paste. Thus upon ingesting, the biochemical affects can help upregulate the endocannabinoid immune system, which then helps provide a biochemical homeostasis, which can then help people self-heal through a daily healthful regimen.

Not only do consumers demand a less expensive Cannabis-Hemp product that can be ingested several times daily, but they also insist the industry adopt a near zero tolerance for pesticide contamination. The liability placed upon Cannabis-Hemp providers, makes it more important than ever, that the retailers provide an organically grown cannabis-hemp product.

Yet, this goal is not entirely attainable in reality. The United States Federal Government hasn't fully recognized Cannabis-Hemp as a nutritional food product. This means that there is no way for a Cannabis-Hemp Grower to get an Organic Certification form the Food and Drug Administration. And this ultimately hurts the industry providers over the long-term.

The present invention enables a single location to process, cure, store, cook and package the Cannabis Hemp Paste food grade nutritional supplement in large quantities. Therefore, each individual retail outlet can eliminate any reliance upon, and the capital expense for, third party quality certifications. They need simply to refer them to the publicly published materials, and the transparent process, being shared by the current patent application owner.

Finally, the novel and useful Cannabis Hemp Paste process utilizes several steps in the production of a final food grade nutritional supplement. Accordingly, an object of the invention is to provide a commercial process for preparing an affordable Cannabis Hemp Paste that can be ingested several times daily, with no harmful side effects.

### DETAILED DESCRIPTION

The present invention provides a commercial process for preparing Cannabis-Hemp Paste. The process produces both a fully cooked and raw food product that can be inserted into an edible consumption vessel of any type.

To process the Cannabis-Hemp Paste, the steps include: providing a quantity of raw cannabis-hemp (flowers, stems, and leaves); providing a means to convey the raw cannabis-hemp product through the various steps of the present invention; adding fats to the raw cannabis-hemp food product; heating and steaming the raw food product with added fats; resting the cannabis-hemp food product (with added fats) after the step of heating and steam-cooking; pressing the cooked cannabis-hemp food product to extract the first pressing of fats (which will be added later in the process); adding fresh fats (after the first pressing) while letting the freshly added fats rest within the cooked and cooling cannabis-hemp food product; recook the previously heated and steamed cannabis-hemp food product (with freshly added fats); press the recooked cannabis-hemp food product to extract the second pressing of fats; add fresh fats (after the second pressing) while letting the freshly added fats rest within the twice cooked and cooled cannabis-hemp food product; rest the twice cooked cannabis-hemp food product (with freshly added fats) after the step of recooking and pressing for the second time; recook the twice cooked cannabis-hemp food product (with freshly added fats); add more fresh fat to the thrice cooked cannabis-hemp food product; add the first and second pressing of fats to the thrice cooked cannabis-hemp food product; add a pressing of the raw cannabis-hemp plant, blend all of the pressings, and freshly added fats, into the thrice cooked cannabis-hemp food product, as well as the raw pressed cannabis-hemp food product; then, if not pasty enough at this point, add a thickening agent (seed meal, flour, beeswax, etc.) and blend; now take the Cannabis-Hemp Paste and add it to an edible consumption vessel of any type (HPMC Capsules), or just put the Cannabis Hemp Paste in your mouth and ingest.

### CANNABIS-HEMP PASTE ABSTRACT RECIPE

Assemble Initial Ingredients:

Organically Grown Fine Ground Cannabis-Hemp Herb (flowers, stems and leaves)

Add Various Fats (Coconut Oil, Walnut Oil, Honey, etc.)

Add Various Thickeners (Seed Meal, Flour, Beeswax, etc.)

### Phase One

Decarboxylized Herb in Convection Oven

Add Fats to Decarboxylized Herb

Steam Kettle Herb with Fats

Extract Fats from Herb with Press

Keep Extracted Fats Resting/Cooled (until final processing)

### A Multi-Phase Process

Phase Two (After First Pressing is Extracted from Herb):

Add fresh fats and let rest/cool

Recook in steam kettle

Extract fats, through second pressing, from recooked herb

Add fresh fats and let rest/cool

Phase Three (After Resting and Cooling for the Second Time)

Recook in steam kettle for third time

After third recooking, add fresh fats

After third recooking, and adding of fresh fats, add first and second pressing of fats

Now press and blend raw cannabis-hemp into the final amalgam

Now let third recooking, and the total re-addition of all food products and pressings rest

Phase Four (After Resting and Cooling for the Third Time)

Now slightly warm the Cannabis-Hemp Paste, then add to edible consumption vessels
if Cannabis Hemp Paste is not ‘pasty’ enough, add a thickening agent (seed meal, flour, wax)

1. A process for preparing ‘cannabis-hemp paste’ comprising the steps of:
a. providing a quantity of the raw cannabis-hemp plant (food product);
b. providing a system to convey the food product through the various steps of the process;
c. heat cooking the food product;
d. blending various fats with the food product;
e. heat cooking the food product with steam;
f. amalgamating the various added fats with the cannabinoids, terpenes, terpenoids, and flavonoids;
g. pressing the cooked and raw food product to extract liquids;
h. blending the extracted liquids;
i. blending the extracted liquids with both the raw and cooked food product.

2. The process of preparing ‘cannabis-hemp paste’ according to claim 1, comprising the further step of injecting the food product with various spices, seeds, nuts, roots, flavorings, condiments, fruits, herbs, flowers, leaves, vegetables, and sweeteners before and after (c, e).

3. The process of preparing ‘cannabis-hemp paste’ according to claim 2, comprising the further step of freezing and or refrigerating the food product before and after each step (a to i).

4. The process of preparing ‘cannabis-hemp paste’ according to claim 2, wherein the step of blending the food product (h, i) comprises the sub-steps of:
(i) applying to the food product a thickener;
(ii) removing liquids from the food product to thicken.

5. The process of preparing ‘cannabis-hemp paste’ according to claim 4, wherein the food product is fully prepared upon exiting the steps of heat and or steam cooking (c, e).

6. A process for preparing ‘cannabis-hemp paste’ comprising the ordered steps of:
a. providing a quantity of the cannabis hemp plant;
b. providing a system to convey the raw food product through the process;
c. heat-cooking the raw food product to decarboxylate a quantity of cannabinoids.
d. blending the cooked food product with fats to provide for different amalgams;
e. resting and cooling the food product, which both cools the food product so that it does not continue to cook, and cools down the food product for handling;
f. heat-cooking the previously cooked and blended food product with steam;
g. pressing the blended and twice-cooked food product to extract a liquid amalgam;
h. adding and blending various fresh fats to the twice cooked food product;
i. resting and cooling the food product;
j. heat-cooking the twice-cooked and blended food product with steam;
k. pressing the blended and thrice cooked food product to extract the liquid amalgam;
l. adding and blending various fresh fats to the thrice cooked food product;
m. pressing a portion of the raw uncooked cannabis-hemp plant to extract liquids;
n. adding and blending all extracted liquids to the thrice cooked and raw food product.
o. Now let the food product/amalgam rest and cool; then gently warm and serve.
